(19) United States
(12) Patent Application Publication
(10) Pub. No.: US 2013/0101643 A1
(43) Pub. Date: Apr. 25, 2013

(54) METHODS AND COMPOSITIONS FOR MITIGATING PROCTITIS

(71) Applicant: Salient Pharmaceuticals Incorporated, Houston, TX (US)

(72) Inventor: Richard M. Scruggs, Houston, TX (US)

(73) Assignee: Salient Pharmaceuticals Incorporated, Houston, TX (US)

(21) Appl. No.: 13/656,518

(22) Filed: Oct. 19, 2012

Related U.S. Application Data
(60) Provisional application No. 61/549,564, filed on Oct. 20, 2011.

(51) Int. Cl. A61K 33/12; A61P 29/00; A61K 9/14

(52) U.S. Cl. 424/400; 424/683

(57) ABSTRACT

The present disclosure relates generally to the field of proctitis. More particularly, methods and compositions for treating, ameliorating and/or preventing proctitis are provided.

### CROSS-REFERENCE TO RELATED APPLICATIONS

The present application claims the benefit of priority to U.S. Provisional Patent Application No. 61/549,564, filed Oct. 20, 2011, which is incorporated herein by reference in its entirety.

### TECHNICAL FIELD

The present disclosure relates to methods and compositions for preventing, treating, ameliorating and/or reducing the severity of proctitis. More particularly, the disclosure relates to methods for preventing, treating, ameliorating and/or reducing the severity of proctitis by providing a composition comprising a clay, and in one embodiment, a calcium-based clay with no appreciable swelling in water.

### BACKGROUND

Proctitis is an inflammation of the anus and the mucosal lining of the rectum, affecting the last 6 inches of the lower intestine known as the rectum. Symptoms of proctitis include ineffectual straining to empty the bowels, diarrhea, rectal bleeding and possible discharge, a feeling of not having adequately emptied the bowels, involuntary spasms and cramping during bowel movements, left-sided abdominal pain, passage of mucus through the rectum, and anorectal pain. Another common symptom is a continual urge to have a bowel movement—the rectum could feel full or have constipation. Subjects having proctitis often have tenderness and mild irritation in the rectum and anal region. A serious symptom is pus and blood in the discharge, accompanied by cramps and pain during the bowel movement. If there is severe bleeding, anemia can also result.

Doctors can diagnose proctitis by looking inside the rectum with a proctoscope or a sigmoidoscope. A biopsy is taken, in which the doctor scrapes a tiny piece of tissue from the rectum, and this tissue is then tested. The physician may also take a stool sample to test for infections or bacteria. If the physician suspects that the patient suffers from Crohn's disease or ulcerative colitis, colonoscopy or barium enema X-rays are used to examine areas of the intestine.

Proctitis has many possible causes. It may occur idiopathically (idiopathic proctitis). Other causes include damage by irradiation (for example in radiation therapy for cervical cancer and prostate cancer) or as a sexually transmitted infection, as in lymphogranuloma venereum and herpes proctitis. Proctitis is also linked to stress and recent studies suggest a celiac disease-associated proctitis can result from an intolerance to gluten (Mazzarella, et al., (2000), Methods Mol Med 41:163-73; Ensari, et al., (1993) Gut 34(9):1225-9).

A common cause is engaging in anal sex with multiple partners infected with sexual transmitted diseases in men who have sex with men (Soni, et al., (2010), Alimentary Pharmacology & Therapeutics 32(1):59-65; Romapalo, et al., (1999) Clinical Infectious Diseases 28(pS84)). Shared enema usage has been shown to facilitate the spread of Lymphogranuloma venereum proctitis (De Vries, et al., (2008), Sexually Transmitted Diseases 35:(2):203-208).

Radiation-induced proctitis is a common side effect of radiation therapy used for the treatment of pelvic cancers including cancers of the prostate, rectum, lower colon, and certain gynecologic malignancies. Overall, about 30% of patients develop significant symptoms associated with anorectal injury. Radiation injury can result in both acute and chronic proctitis. The most common symptom is a frequent or continuous sensation or urge to have a bowel movement or frank fecal incontinence. Other symptoms include constipation, a feeling of rectal fullness, left-sided abdominal pain, passage of mucus through the rectum, rectal bleeding, and anorectal pain (Wong, et al., (2003) Int. J. Radiat. Oncol. Biol. Phys. 55(5):1254-64).

The development of chronic proctitis is especially bothersome. No effective interventions are currently available (Mangar et al., (2005) Eur. J. Cancer 41(6):908-21). Treatment for proctitis varies depending on severity and the cause. Standard therapy for proctitis is predominantly palliative and focused on pain control and maintenance of nutrition. However, recent data indicates that even opiods are often insufficient to control proctitis pain. A physician may prescribe antibiotics for proctitis caused by bacterial infection. If the proctitis is caused by Crohn's disease or ulcerative colitis, the physician may prescribe the drug 5-aminosalicyclic acid (5ASA) or corticosteroids applied directly to the area in enema or suppository form. Enema and suppository applications may more effective, but some patients may require a combination of enema and suppository applications. Another treatment available is that of fiber supplements such as Metamucil. Taken daily these may restore regularity and reduce some of the pain associated with proctitis.

Given that a large number of patients suffer proctitis annually and patients undergoing cancer therapy often receive multiple cycles of chemotherapy and/or radiation therapy, there is a significant need for improved treatment of radiation induced proctitis. The present disclosure is directed to this need.

### BRIEF SUMMARY

The following aspects and embodiments thereof described and illustrated below are meant to be exemplary and illustrative, not limiting in scope.

In one aspect, a method of treating proctitis is provided, the method comprising administering to a subject a therapeutically effective amount of a composition comprising a clay.

In another aspect, a method to reduce or delay the onset of proctitis is provided, the method comprising administering to a subject a therapeutically effective amount of a composition comprising a clay.

In yet another aspect, a method for preventing proctitis in a subject is provided, the method comprising administering to a subject a therapeutically effective amount of a composition comprising a clay.

In still another aspect, a method for reducing severity of proctitis in a subject is provided, the method comprising administering to a subject a therapeutically effective amount of a composition comprising a clay.

In one embodiment, the clay for use in any of the methods described herein is a low-swelling or a non-swelling, calcium species of clay.

In one embodiment, the clay is a smectite clay.

In another embodiment, the clay is a calcium montmorillonite clay. In one embodiment, the montmorillonite clay comprises a majority of particles with a dry state fractionation size of between about 50-200 μm. In another embodiment, the montmorillonite clay comprises a majority of particles with a wet state fractionation size of less than 2 μm. In a specific embodiment, the montmorillonite clay is non-swelling and a calcium species as evidenced by a shrink/swell potential (SV) of less than 1.5 COLE index value. In yet another embodiment, the montmorillonite clay exhibits an extractable bases value for calcium of greater than 90 mEq/100 g clay, when extracted using ammonium acetate, or a value for calcium of between 6-12 mEq/100 g clay/L when extracted with deionized water.

In yet another embodiment, the clay has a uniform particle size that is achieved by sieving or air classification of the clay.

The method, in one embodiment, is provided to a subject with proctitis. In other embodiments, the subject is one at risk of developing proctitis.

The method, in another embodiment, is provided to a subject with radiation-induced proctitis. The method, in another embodiment, is provided to a subject with chemotherapy-induced proctitis.

The method, in another embodiment, is provided to a subject with cancer.

In other embodiments, the method is provided to a subject undergoing or planning to undergo chemotherapy.

In still other embodiments, the method is provided prior to or concurrent with initiation of radiation therapy in the cancer subject.

In yet other embodiments, the method comprises administering after radiation therapy. Alternatively, the method can comprise administering for the duration of radiation therapy.

In other embodiments, the method comprises administering the clay composition more than once daily.

In one embodiment, the composition is administered rectally as a fluid comprising the clay. In one embodiment, the composition is administered orally as a fluid comprising the clay. The fluid can be, for example, a solution, a suspension, a paste, or a gel.

In other embodiments, the clay-containing composition further comprises a polymer. In one embodiment, the polymer is a bioadhesive polymer.

In still other embodiments, the clay containing composition comprises a solid dosage form that disintegrates in an aqueous medium. In one embodiment, the aqueous medium is a body fluid.

In other embodiments, the subject receiving the treatment method is concurrently treated with at least one therapeutic agent. In exemplary embodiments, the therapeutic agent is a pain reliever or a chemotherapeutic; an anti-inflammatory or antibiotic. In one embodiment, the pain reliever is a topical anaesthetic selected from the group consisting of fentanyl, hexylresorinol, dyclonine hydrochloride, asbenzocaine and phenol. In a preferred embodiment, the pain reliever is fentanyl.

In some embodiments, the composition for administration is provided in form such that prior to administering, the clay is contacted with a fluid to form a composition suitable for rectal or oral administration.

In some aspects, the present disclosure provides methods for treating, ameliorating or preventing proctitis comprising administering to a subject a therapeutically effective amount of a composition comprising a clay. Patients to be treated according to the disclosed methods and compositions include those who have proctitis, including radiation-induced or chemotherapy-induced proctitis. In addition, patients who do not have, but are at risk of developing, proctitis can be treated according to the present disclosure. In the latter group of patients, the treatment can inhibit, delay or prevent the development of proctitis. In some embodiments, the patient to be treated is a subject having cancer. In some embodiments, the subject to be treated is suffering from proctitis or is at risk of developing proctitis. In some embodiments, the proctitis to be treated, ameliorated or prevented is proctitis. In some embodiments, the proctitis to be treated, ameliorated or prevented is radiation-induced proctitis. In some embodiments, the proctitis to be treated, ameliorated or prevented is chemotherapy-induced proctitis.

In certain embodiments, the subject has received or will be receiving radiation therapy or chemotherapy. In certain embodiments, the proctitis is caused or is likely to result from radiation-induced toxicity in non-malignant tissue. In other embodiments, the proctitis is caused or is likely to result from chemical-induced toxicity in non-malignant tissue. In one embodiment, the chemical-induced toxicity is not caused by docetaxel.

In one embodiment, the subject to be treated is a bone marrow transplantation patient. In another embodiment, the subject to be treated is a cancer patient. The subject may have any type of cancer. In certain embodiments, the subject has leukemia, lymphoma, rectal or colorectal cancer, breast cancer, prostate cancer, androgen-dependent prostate cancer, lung cancer, mesothelioma, pelvic cancer, gastric cancer, pancreatic cancer, gastrointestinal cancer, renal cell cancer, testicular cancer, germ cell cancer, glioma or any other primary or solid tumor. In one embodiment, the subject does not have androgen-independent prostate cancer.

Examples of treatments that may cause or place a patient at risk of developing proctitis are radiation therapy and/or chemotherapy, as described further elsewhere herein or in the background section. Patients that can be treated according to the present disclosure thus include, for example, cancer patients, HIV/AIDS patients, as well as patients that have recently been, will shortly be, or are currently subject to treatment with pelvic irradiation, or stem cell or bone marrow transplantation.

According to the disclosed methods, compositions used herein can be administered to a patient prior to, concurrently with, or after a treatment that has induced or places the patient at risk of developing proctitis, or a combination of these approaches can be used. In an example, the composition is administered at the same time as, within 1-4 hours of, or on the same day as the treatment, and then for 1-3 (e.g., 1-2) days thereafter (e.g., 1-2 times per day). Other examples of treatment regimens are provided below.

The compositions can be administered to patients by any acceptable manner known in the art, including topically (e.g., by gel, rinse, lozenge, cream, ointment, or patch), orally (e.g., by tablet, capsule, lozenge, cream, ointment, or patch), rectally (e.g., by suppository, ointment, or enema), or vaginally (e.g., by cream, ointment, gel, or suppository). Also, treatment according to the present disclosure can be carried out in combination with other approaches to treating proctitis, including antimicrobial and palliative treatments, as is discussed further below.

In some embodiments, the subject is concurrently treated with at least one therapeutic agent. In some embodiments, the therapeutic is a pain reliever or a chemotherapeutic. In some embodiments, the therapeutic agent is an anti-inflammatory or antibiotic. In some embodiments, the pain reliever is a topical anaesthetic selected from, but not limited to, the group consisting of fentanyl, hexylresorinol, dyclonine hydrochloride, asbenzocaine and phenol. In some embodiments, the pain reliever is fentanyl, hexylresorinol, dyclonine hydrochloride, asbenzocaine and phenol. In some embodiments, the administering comprises administering to a subject undergoing or planning to undergo chemotherapy. In some embodiments, the administering is prior to or concurrent with initiation of radiation therapy in the cancer subject. In some embodiments, the administering is after radiation therapy. In some embodiments, the administering continues for the duration of radiation therapy. In some embodiments, the administering continues for longer than the duration of radiation therapy. In some embodiments, the administering continues for a time shorter than the duration of radiation therapy. In some embodiments, the administering comprises administering more than once daily. In some embodiments, the administering comprises administering once daily.

In some embodiments, the composition used in the method does not include vitamin D. In some embodiments, the therapeutic agent is not vitamin D. In some embodiments, the patient is not being or has not been treated with vitamin D.

Further, the present disclosure includes compositions including the compounds described herein, formulated for administration for reducing the severity of proctitis as described herein. As is described in detail below, these compositions can include the compounds in formulations such as gels for topical administration, rinses, tablets, capsules, lozenges, creams, ointments, enemas, suppositories, or patches. In some embodiments, the compositions is administered topically, to a mucous membrane. In some embodiments, the composition is administered intrarectally. In some embodiments, the composition is administered orally.

In some embodiments, the method further comprises squirting the fluid into the rectum. In some embodiments, the administering comprises administering the suppository into the rectum for a recommended period of time.

In addition to the exemplary aspects and embodiments described above, further aspects and embodiments will become apparent by reference to the drawings and by study of the following descriptions.

### BRIEF DESCRIPTION OF THE FIGURES

FIG. 1 shows the mean percentage weight change as a function of day in animals with induced proctitis;

FIG. 2 is a bar graph showing the area under the curve (AUC) calculated for the percentage weight change in the animals of FIG. 1;

FIG. 3 presents the mean endoscopy proctitis score on day 3;

FIG. 4 presents the mean endoscopy proctitis score on day 7;

FIG. 5 presents the mean endoscopy proctitis score on day 10;

FIG. 6 shows the mean endoscopy proctitis score for days 3 to 10;

FIG. 7 shows representative endoscopy images from each treatment group;

FIG. 8 shows the percentage of animals exhibiting diarrhea in each treatment group, over the course of one study; and

FIGS. 9A-9B are bar graphs showing the amount, in pg/mL of TNF-α present in solution after incubation with the noted concentrations of calcium aluminosilicate clay (FIG. 9A) and the percent of TNF-α relative to the control solution containing no clay for each of the clay solutions (FIG. 9B).

Various aspects now will be described more fully hereinafter. Such aspects may, however, take many different forms and should not be construed as limited to those embodiments explicitly set forth herein. Rather, these embodiments are provided so that this disclosure will be thorough and complete, and will fully convey its scope to those skilled in the art.

### DETAILED DESCRIPTION

### I. DEFINITIONS

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

It must be noted that, as used in this specification, the singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

As used herein, “proctitis” refers to the inflammation ulceration of the mucous membranes, more specifically of mucosal epithelium of the anus and rectum, especially following chemotherapy or radiotherapy in patients suffering from tumors and to the symptomatic features associated therewith, i.e. pain, redness, inflammation, ulceration, or combinations thereof, affecting the mucosal epithelium of the rectum. Proctitis can result from disease or can arise secondarily to therapeutic treatments such as certain chemotherapies, radiation, or combinations thereof, or to any proctitis-inducing circumstance or event.

The term “proctitis” shall also include colitis, and intends inflammation and/or destruction of the mucosal lining of all or a portion of the colon, caecum, rectum and/or anus.

As used herein, the phrase “therapeutically effective amount” refers to an amount which provides a therapeutic benefit, wherein benefits can include, the prevention, treatment or amelioration of proctitis.

### II. METHODS OF TREATMENT

In a first aspect, a method for preventing, treating, ameliorating and/or reducing the severity of proctitis is provided. As will be illustrated by the studies discussed herein, administration of a composition comprising a clay is effective in treating, slowing the progression of, reducing the severity of, and preventing proctitis. These studies are described in Section A. In Section B, clays and formulations comprising a clay for use in the treatment method are described, and in Section C, treatment regimens in general and for particular patient populations are disclosed.

### A. Treatment of Proctitis

Studies were conducted in support of the claimed methods to demonstrate the efficacy of a composition comprising a clay to prevent proctitis, treat proctitis, ameliorate the severity of proctitis and/or reduce the severity of proctitis. As described in Example 1, an acute radiation model was used wherein colitis was induced in male rats. In the study, an acute radiation dose of 20 Gy was administered to each animal. Clinically significant proctitis was observed on days 3 through 10.

In this model of proctitis, male rats were anesthetized by the injection of ketamine and xylazine before being placed under the radiation source. Lead shielding was used to protect all of the body, excluding a 1 inch strip immediately above and including the anus. This procedure had been previously validated and described (Kang, et al., (2000) J. Korean Med. Sci. 15:682-689; Korkut, et al., (2006) World J. Gastroenterol. 12:4879-4883; Northway, et al., (1988) Cancer 62:1962-1969). Animals received an acute radiation dose of 20 Gy targeted to the lower abdomen on day 0. This results in a peak of proctitis on day 7 or day 8. To evaluate colitis severity, animals were anesthetized with an inhalation anesthetic, and underwent video endoscopy of the lower colon on days 3, 7, and 10. Proctitis was scored visually on a scale that ranges from 0 for normal, to 4 for severe ulceration. Following endoscopy on day 10, all remaining rats were sacrificed and tissue is taken for histology. The colon was removed, measured, and weighed and then fixed in 10% neutral buffered formalin.

Twenty-five rats were prospectively randomized into two treatment groups of ten (10) animals each and a control group of five (5) animals. On study day 0, twenty rats were anesthetized with ketamine (100 mg/kg) and xylazine (5 mg/kg) and placed beneath 4 mm lead shields with their lower abdomens exposed. Proctitis was induced on Day 0 by a single acute dose of radiation of 20 Gy directed to the lower abdomen. A group of 5 animals served as un-irradiated controls. All animals were assessed visually daily for the presence of diarrhea and/or bloody stool and body weights were measured once daily from Day −3 to Day 10. On days 3, 7, and 10 proctitis severity was assessed in all animals using video endoscopy, and images were taken and proctitis severity scored by a blinded observer. A composition comprising clay was given intrarectally (i.r.) twice daily (b.i.d.) from day 0 to day 10 while another group was administered water for injection (WFI) for the same schedule, as indicated in Table 1. As a control, animals in Group I received a saline vehicle twice daily at the same dose volume as the clay compositions. The clay selected for use in the study was a calcium-based aluminosilicate clay, which is essentially non-swelling in aqueous fluids. The clay is discussed in more detail hereinbelow. On day 10, all animals were euthanized by CO2 asphyxiation and the colon and rectal tissues were dissected and fixed in neutral buffered formalin for histological evaluation.

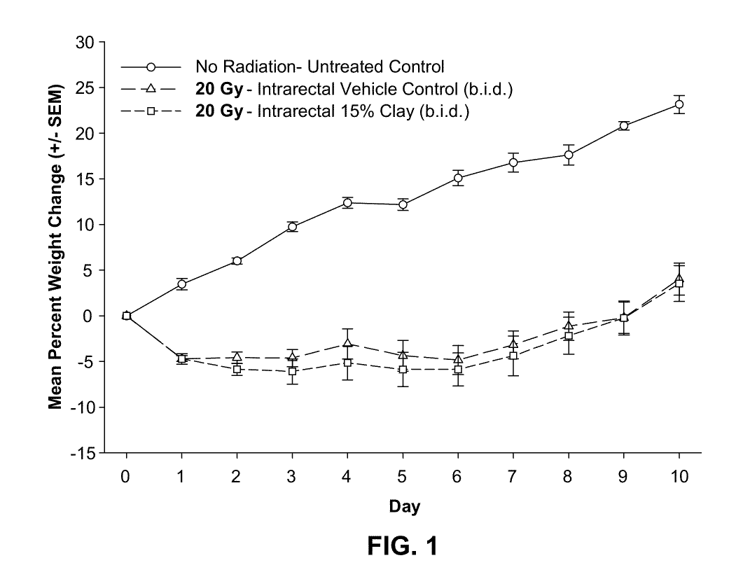

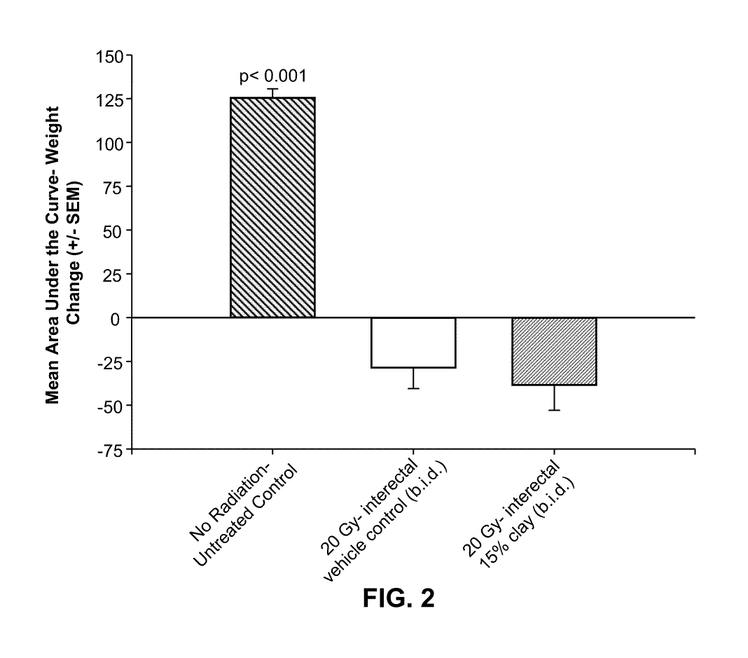

All rats were weighed daily and their survival recorded in order to assess possible differences in animal weight among treatment groups as an indication for proctitis severity and/or possible toxicity resulting from the treatments. The mean daily percent weight change of the test animals is presented in FIG. 1. The statistical significance of these differences was evaluated by calculating the area under the curve (AUC) using the trapezoidal rule transformation for the weight gain of each animal, and then comparing the different treatment groups using a One-Way ANOVA test. The results of this analysis indicated that there were no significant differences between any of the treatment groups. These data are shown in FIG. 2 and Table 1, below.

TABLE 1

| | | | | | Video | |
|---|---|---|---|---|---|---|
| | Number of | | | Dosing | Endoscopy | |
| Group | Animals | Radiation | Treatment | Schedule | Schedule | Volume |
| 1 | 5 | NO | Untreated | — | Days | — |
| | | | | | 3, 7, 10 | |
| 2 | 10 | 20 Gy | Vehicle-WFI | Days 0 | Days | 0.5 mL/Dose |
| | | Day 0 | (intrarectal) | to 10* | 3, 7, 10 | |
| | | | | (BID) | | |
| 3 | 10 | 20 Gy | 15% clay | Days 0 | Days | 0.5 mL/Dose |
| | | Day 0 | (intrarectal) | to 10* | 3, 7, 10 | |
| | | | | (BID) | | |

*The first dose of clay on Day 0 was administered 1-2 hours following radiation.

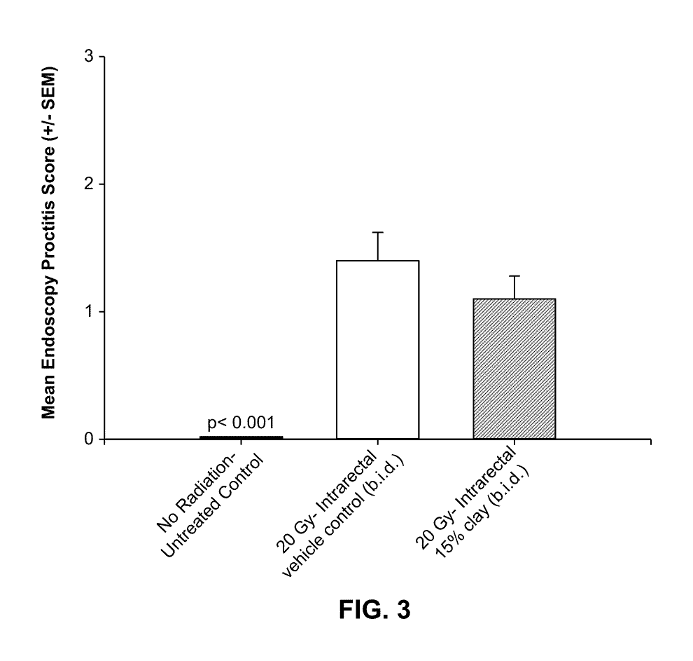

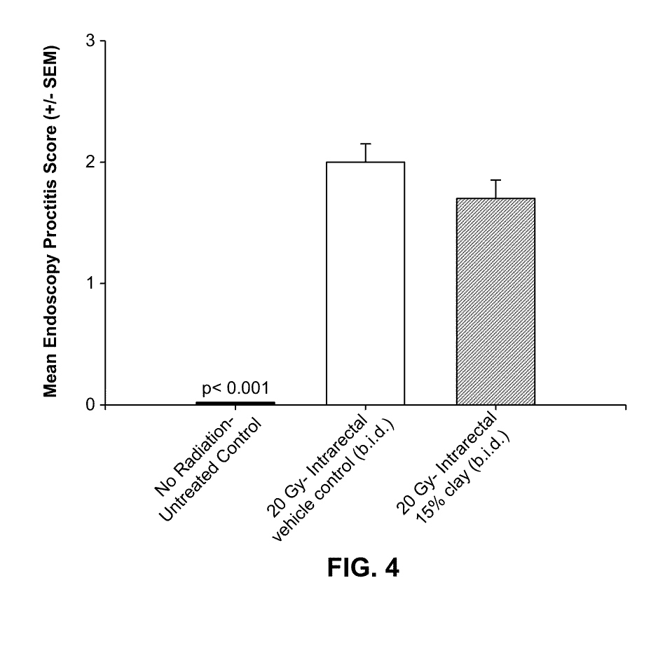

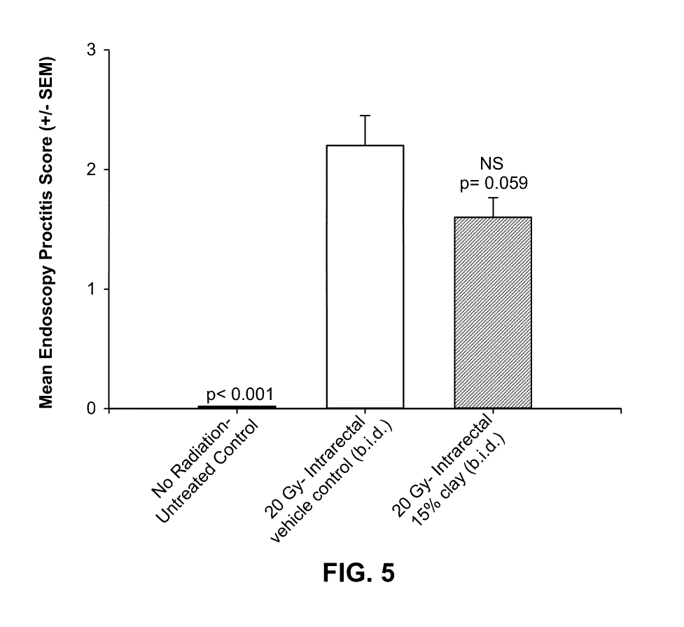

All animals underwent video endoscopy on days 3, 7 and 10 to assess the severity of proctitis in each treatment group. The mean proctitis scores for all treatment groups on the individual days are shown in FIGS. 3-5. Proctitis scores were significantly elevated in the radiation group treated with only vehicle compared to the untreated control group on all days of endoscopy.

Proctitis was scored visually by comparison to a validated photographic scale, ranging from 0 for normal, to 4 for severe ulceration (clinical scoring). In descriptive terms, this proctitis scoring scale is defined in Table 2-1, below.

TABLE 2
Endoscopy Colitis Scoring Scale

| Score: | Description: |
|---|---|
| 0 | Normal |
| 1 | Loss of vascularity |
| 2 | Loss of vascularity and friability |
| 3 | Friability and erosions |
| 4 | Ulcerations and bleeding |

Treatment with 15% clay substantially reduced the extent of proctitis, especially apparent on Day 10, however this improvement just missed reaching statistical significance (p=0.059).

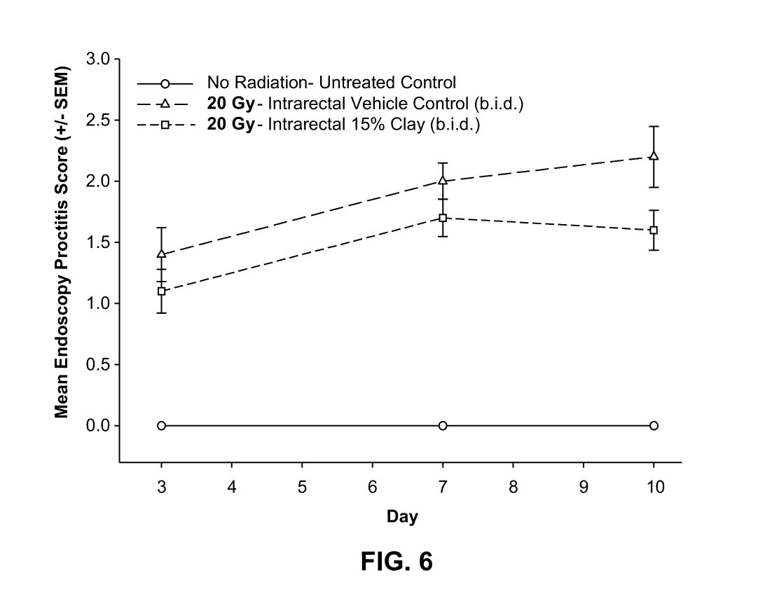

Specifically, on day 3, the untreated control group had an average proctitis score of 0.0, while the radiation-treated control group had an average score of 1.4. The group treated with 15% clay had an average score of 1.1. On day 7, the untreated control group had an average proctitis score of 0.0, while the radiation-treated control group had an average score of 2.0 and the group treated with 15% clay had an average score of 1.7. On day 10, the untreated control group maintained an average proctitis score of 0.0, while the radiation-treated control group had an average score of 2.2 and the group treated with 15% clay had an average score of 1.6. Significant improvements in the treatment groups were assessed through t-test comparison to the radiation treated vehicle control group. FIG. 6 simply shows all endoscopy scores from the three treatment groups on each day of the study.

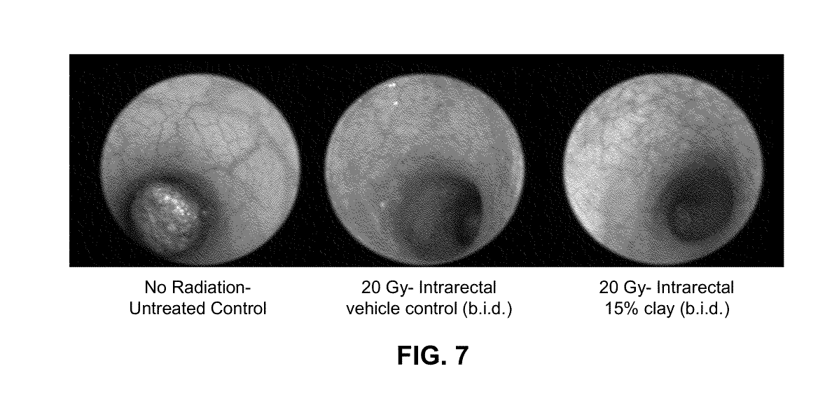

The treatment responses of the colon/rectum to the radiation and clay treatment is readily apparent in the images presented in FIG. 7. The corresponding endoscopy scores for the images presented in FIG. 7 are as follows: untreated control—score of 0; radiation vehicle control—score of 2, inflammation, altered vascularity and friability; clay—score of 1, some altered vascularity.

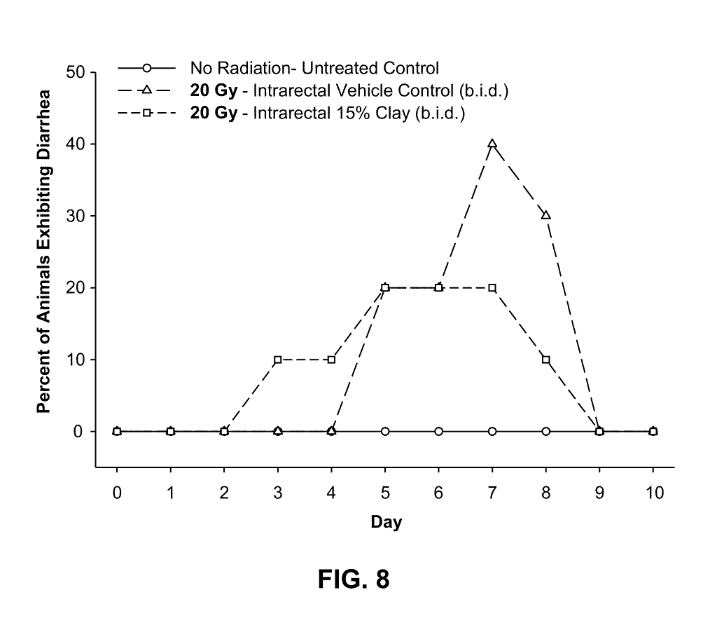

### Diarrhea (FIG. 8).

The percentage of animals exhibiting diarrhea on each study day for all treatment groups are shown in FIG. 8. The vehicle control group exhibited peak levels of diarrhea on Day 7, with 40% of the animals exhibiting diarrhea. Levels of diarrhea were notably lower in the clay treatment group, as diarrhea was only exhibited by 20% of the animals in this group from Days 5 to 7.

No adverse reactions to treatment with clay, given twice daily, were observed over the duration of the study.

As anticipated, the radiation had a significant negative impact on the weight gain of all groups receiving radiation. There was a statistically significant increase in weight change between the non-irradiated controls and the vehicle-treated radiation control group. There was no statistically significant difference in weight change in the group that received 15% clay as compared to the radiation control group.

Radiation induced significant levels of proctitis in the vehicle control group when compared to the untreated control group on all days of endoscopy. Treatment with 15% clay via intrarectal administration resulted in near significant improvements in proctitis severity.

The vehicle control group exhibited peak levels of diarrhea on Days 7 and 8 (40 and 30% of animals, respectively). Levels of diarrhea were notably lower in the clay treatment group, as peak levels reached only 20% of the animals in this group from Days 5 to 7.

In another study conducted in support of the claimed methods, the binding of cytokines to the calcium aluminosilicate clay was investigated. In this study, a fixed concentration of cytokine was exposed to varying concentrations of clay for 15 minutes at room temperature. The samples were stirred occasionally and then centrifuged. The concentration of protein was determined using a sandwich immunoassay kit (R&D Systems, Minneapolis, Minn.) and compared to that measured in a sample without clay. Results are shown in Table 3.

TABLE 3

Clay Concentration
(mg/mL)

| | | | |
|---|---|---|---|
| 0.01 | 0.1 | 1.0 | 10.0 |

| | | |
|---|---|---|
| | Concentration | Percent of Cytokine |
| Material | (pg/mL) | Bound by Clay |

| | | | | | |
|---|---|---|---|---|---|
| IL-1β | 750 | 40 | 52 | 71 | 80 |
| IL-6 | 750 | 35 | 41 | 45 | 89 |
| IL-8 | 200 | 0 | 29 | 43 | ND |
| TNF-α | 750 | 5 | 61 | 90 | 99 |

The data in Table 3 shows that pro-inflammatory cytokines such as TNF-alpha are readily bound by the clay. At a concentration of 1 mg/mL clay, 90% of the TNF-alpha was removed from solution presumably by direct binding to the clay. Binding IL-1beta by the clay was moderate while binding of IL-6 and IL-8 was weak. The unique ability of the clay to bind TNF-alpha was further investigated, as blocking the action of TNF can be beneficial in reducing the inflammation in proctitis and in other diseases. The study is described in Example 3, and the data is presented in FIGS. 3A-3B. The data presented in the two graphs demonstrates the removal of TNF-alpha from a stock solution of this pro-inflammatory protein. Even concentrations of the calcium aluminosilicate clay as low as 0.5 mg/ml removed greater than 90% of all TNF-α from solution. The study demonstrates the capacity to bind and remove from solution important inflammatory components such as tumor necrosis factor and, also illustrates the small amount of clay needed to achieve a benefit. The later type of information is of direct relevance in determining appropriate doses for clay compositions.

Accordingly, in one embodiment a composition comprising a clay to prevent proctitis, treat proctitis, ameliorate the severity of proctitis and/or reduce the severity of proctitis is provided to a subject in need. The ability of the clay to alter the pro-inflammatory environment in the tissue area is demonstrated by the data, and the efficacy of the clay to prevent and ameliorate proctitis is evident from the animal studies.

### B. Clay and Formulations Comprising Clay

The methods of treatment comprise providing to a patient, for administration by a suitable route depending on the type of proctitis, the extent of tissue damage and location of tissue damage, a composition comprising the clay.

### B1. Clay

Clays are a distinguished from other fine-grained earth deposits, and are grouped distinct classes such as kaolinites, illites, smectites. Clays based on silicates form a large class, where the basic structural unit for silicate clays is a SiO4 tetrahedron in which Si⁴⁺ is located at the center and four O²⁻ are positioned at the apices. The tetrahedral structures can be linked together by sharing four O²⁻ ions and together can form a variety of more complex structures including rings (cyclosilicates), chains (inosilicates), sheets (phyllosilicates) and three dimensional arrangements (tectosilicates). Tetrahedra, e.g., SiO₄ and octahedral, e.g., Al₂O₃ are common structural components in many mineral structures.

In one embodiment, the clay for use in the methods described herein is a phyllosilicate clay. Phyllosilicate clays contain both tetrahedral and octahedral sheets, and are further categorized according to composition and packing arrangement. In some embodiments, the clay material is a phyllosilicate selected from the group consisting of kanemite, kenyaite, magadiite and makatite. In some embodiments, the phyllosilicate is selected from the group consisting of allophane (hydrated aluminum silicate); apophyllite (hydrated potassium sodium calcium silicate hydroxide fluoride); bannisterite (hydrated potassium calcium manganese iron zinc aluminum silicate hydroxide); carletonite (hydrated potassium sodium calcium silicate carbonate hydroxide fluoride); cavansite (hydrated calcium vanadate silicate); chrysocolla (hydrated copper aluminum hydrogen silicate hydroxide); clay minerals (described in detail below); delhayelite (hydrated sodium potassium calcium aluminum silicate chloride fluoride sulfate); elpidite (hydrated sodium zirconium silicate); fedorite (hydrated potassium sodium calcium silicate hydroxide fluoride); franklinfurnaceite(calcium iron aluminum manganese zinc silicate hydroxide); franklinphilite (hydrated potassium manganese aluminum silicate); gonyerite(manganese magnesium iron silicate hydroxide); gyrolite (hydrated calcium silicate hydroxide); kanemite; kenyaite; leucosphenite (hydrated barium sodium titanium boro-silicate); magadiite; makatite; micas such as biotite(potassium iron magnesium aluminum silicate hydroxide fluoride), lepidolite(potassium lithium aluminum silicate hydroxide fluoride), muscovite(potassium aluminum silicate hydroxide fluoride), paragonite(sodium aluminum silicate hydroxide), phlogopite(potassium magnesium aluminum silicate hydroxide fluoride) and zinnwaldite(potassium lithium aluminum silicate hydroxide fluoride); minehillite (hydrated potassium sodium calcium zinc aluminum silicate hydroxide); nordite(cerium lanthanum strontium calcium sodium manganese zinc magnesium silicate); octosilicate; pentagonite (hydrated calcium vanadate silicate); petalite(lithium aluminum silicate); prehnite(calcium aluminum silicate hydroxide); rhodesite (hydrated calcium sodium potassium silicate); sanbornite(barium silicate); serpentines such as antigorite(magnesium iron silicate hydroxide), clinochrysotile (magnesium silicate hydroxide), lizardite(magnesium silicate hydroxide), orthochrysotile (magnesium silicate hydroxide) and serpentine(iron magnesium silicate hydroxide); wickenburgite (hydrated lead calcium aluminum silicate); and zeophyllite (hydrated calcium silicate hydroxide fluoride).

In a preferred embodiment, the clay is a dioctahedral smectite, calcium aluminosilicate clay. Calcium aluminosilicate clay is a 2:1 phyllosilicate clay containing sheets of 6-membered rights, and is a very pure calcium montmorillonite clay in the dioctahedryl smectite group. Its general chemical formula is (Na, Ca)_{0.3}(Al, Mg)₂Si₄O₁₀(OH)₂ n(H₂). This clay is a ‘non-swelling’ clay due to the fact it contains more calcium than sodium. As used herein, essentially “low-swelling” and essentially “non-swelling” clay species means that the particular clay species has minimal capacity for changes in volume due to shrinkage or swelling. One approach to measure swelling potential is given in the Soil USDA Technical Handbook, where a COLE index value is assigned to materials. A COLE index value >0.03 indicates that a material may have a small amount of smectite clay within its composition and show low shrink/swell potential. Sodium montmorillonite clays swell much more in aqueous fluids than calcium montmorillonte clays. In one embodiment, the clay for use in the methods described herein is a montmorillonite clay that is non-swelling and a calcium species, as evidenced by a shrink/swell potential (SV) of less than 1.5 COLE index value. An exemplary non-swelling calcium clay is described in U.S. Patent Application Publication No. 2008/0026079, which is incorporated by reference herein.

Due to the presence on the surface of many functional groups, clays are able to interact with other components of the formulation. Negative charges on the clay particles are compensated by counterions, for example Na+, Ca++, Ag+, and so forth, or a combination thereof. Cation exchange capacity (CEC) is an intrinsic property defining the concentration of negatively charged sites on clay particles. Cation exchange capacity is a measure of exchangeable bases in the clay material, and provides an indication of the capacity of the clay to exchange/interact with other compounds. The cationic exchange capacity of clays can be measured using the method described in Grimshaw, The Chemistry and Physics of Clays, Interscience Publishers, Inc., pp. 264-265 (1971). Another common method for determining CEC uses 1 M ammonium acetate (NH₄OAc) at pH 7 (neutral NH₄OAc) and is a standard method used for soil surveys by the Natural Resource Conservation Service. In one embodiment, the clay for use in the methods described herein exhibits an extractable bases value for calcium, as determined using the latter-mentioned method, of greater than 90 mEq/100 g clay, when extracted using ammonium acetate, or a value for calcium of between 6-12 mEq/100 g clay/L when extracted with deionized water.

A skilled artisan will appreciate that clay can be sized by sieving or by air classification to achieve a desired average particle size. A skilled artisan in the relevant field will appreciate that any representative sample of clay will be polydisperse in size, yet the art provides several approaches for expressing the average size, or diameter, of particles in a population. For example, an average particle size, or uniform particle size, may in some embodiments intend that a representative sample of the clay when passed through a sieve of a certain mesh size retains a majority (greater than 50%) of the clay sample. Size fractionation (wet and dry) of a representative sample of a given clay can be done as follows. Wet state fractionation involves removal of cementing and flocculating materials in the sample using sodium acetate buffer (pH 5). Organic matter in the sample is removed using 30% hydrogen peroxide. The sample is then dispersed with 50 mL of pH 10 sodium carbonate solution. The sand fraction (>53 μm) is separated using a 53 μm sieve. The clay fraction (<2 μm) is separated from the silt fraction (2-53 μm) by centrifugation using pH 10 sodium carbonate as dispersant. Sand and silt weights are recorded after drying at 105° C. overnight. Clay suspension is flocculated with sodium chloride and then dialyzed until the electrical conductivity measurement were close to the values of deionized water (<2 pS/cm). Air dry fractionation of clay can be done using, for example, an Octagon 200 sieve shaker (Endecotts), and weight average particle size determined in accord with the manufacturer's instructions. For example, the percentage of particles with greater than 100 μm size, between 45-100 μm size, and less than 45 μm size is calculated. In one embodiment, the montmorillonite clay comprises a majority (i.e., 51%, based on weight percent) of particles with a dry state fractionation size of between about 50-200 μm. In other embodiments, the montmorillonite clay is comprised of a particles wherein at least 60%, 70%, 75% or 80% of the particles have a dry state fractionation size of between about 50-200 μm. In another embodiment, montmorillonite clay comprises a majority of particles with a wet state fractionation size of less than 2 μm. In another embodiment, the average particle size of the clay in a dry state is less than about 200 μm, or less than about 100 μm, or less than about 80 μm. In other embodiments, the average particle size of the clay is between 5-200 μm or between 5-50 μm.

Suitable clay minerals include chlorites such as baileychlore(zinc iron aluminum magnesium silicate hydroxide), chamosite(iron magnesium aluminum silicate hydroxide oxide), the generalized mineral chlorite, clinochlore (a chromium variety kaemmererite) (iron magnesium aluminum silicate hydroxide), cookeite(lithium aluminum silicate hydroxide), nimite(nickel magnesium iron aluminum silicate hydroxide), pennantite(manganese aluminum silicate hydroxide), penninite(iron magnesium aluminum silicate hydroxide) and sudoite(magnesium aluminum iron silicate hydroxide); glauconite(potassium sodium iron aluminum magnesium silicate hydroxide); illite (hydrated potassium aluminum magnesium iron silicate hydroxide); kaolinite(aluminum silicate hydroxide); montmorillonite (hydrated sodium calcium aluminum magnesium silicate hydroxide); palygorskite (hydrated magnesium aluminum silicate hydroxide); pyrophyllite(aluminum silicate hydroxide); sauconite (hydrated sodium zinc aluminum silicate hydroxide); talc(magnesium silicate hydroxide); and vermiculite (hydrated magnesium iron aluminum silicate hydroxide).

For example, palygorskite or attapulgite is a magnesium aluminium phyllosilicate with formula (Mg,Al)₂Si₄O₁₀(OH).4(H₂O) which occurs in a type of clay soil common to the Southeastern United States. Attapulgite clays are a composite of smectite and palygorskite. Smectites are expanding lattice clays of which bentonite is a commonly known generic name for smectite clays. The palygorskite component is an acicular bristle-like crystalline form which does not swell or expand. Attapulgite forms gel structures in fresh and salt water by establishing a lattice structure of particles connected through hydrogen bonds. Attapulgite, unlike bentonite, will form gel structures in salt water and is used in special salt water drilling mud for drilling formations contaminated with salt. Palygorskite particles can be considered as charged particles with zones of + and − charges. It is the bonding of these alternating charges that allow them to form gel suspensions in salt and fresh water. Attapulgite clays found in the Meigs-Quincy district are bundles of palygorskite clay particles between 2 and 3 micrometres long and below 3 nanometres in diameter. The bundles are surrounded by a matrix of smectite clays which are slightly swellable. Dry process grades contain up to 25% non-attapulgite material in the form of carbonates and other mineral inclusions.

Kaolin is not as absorbent as most clays used medicinally and has a low shrink-swell capacity. Also, it has a low cation exchange capacity. This clay is also known as ‘white cosmetic clay’. Clay, in the form of kaolin, is still a common ingredient in western medicines such as Rolaids and Maalox, as well as in cosmetics.

Swellable clay minerals are typically those that have alkali metals between their layers and can swell in polar solvents. These include lithium containing materials such as cookeite; sodium containing materials such as glauconite (which also contains potassium), montmorillonite and sauconite; and potassium containing materials such as illite.

In some instances, non-swellable materials are preferred over the swellable clay minerals.

It may be desirable to treat the phyllosilicate particles with an organic material to intercalate organic molecules between adjacent, planar silicate layers. For example, treatment can be with an organic material such as silane coupling agents; quaternary ammonium compounds; monomeric compounds having an electrostatic functionality selected from the group consisting of amines, amides and mixtures thereof; monomeric compounds having a functionality selected from the group consisting of hydroxyl, aromatic rings, carbonyl, carboxylic acid, polycarboxylic acid, aldehydes, ketones, amines, amides, ethers, esters and combinations thereof; and so forth.

### B2. Formulations Comprising Clay

The clay is formulated with one or more pharmaceutical excipients to provide a composition for administration to a patient in need of treatment. Compositions contemplated for use in the methods are varied according to these, and other, factors, and exemplary formulations are now described.

In a first embodiment, a composition suitable for oral administration to a patient in need is contemplated. The composition comprises an amount of clay sufficient to achieve a desired therapeutic response when administered to the patient in accord with a defined protocol. Formulation of the clay into a form suitable for oral administration includes formulation to provided, for example, a liquid formulation such as a suspension, a solution, an elixir, or an emulsion, that can be used as a mouth rinse, spray or wash. Liquid formulations often are aqueous based and can include excipients to increase the viscosity to provide a coating on the mucosal tissue that lingers for a period of time after application. Mouthwash formulations are well-known to those skilled in the art and will often include excipients as, for example, pharmaceutical grades of mannitol, lactose, starch, magnesium stearate, sodium saccharine, cellulose, magnesium carbonate and/or the like. Exemplary formulations are discussed in detail, for example, in U.S. Pat. No, 6,387,352, U.S. Pat. No. 6,348,187, U.S. Pat. No. 6,171,611, U.S. Pat. No. 6,165,494, U.S. Pat. No. 6,117,417, U.S. Pat. No. 5,993,785, U.S. Pat. No. 5,695,746, U.S. Pat. No. 5,470,561, each of which is herein specifically incorporated by reference into this section of the specification and all other sections of the specification.

Oral formulations can also take the form of a lozenge, a treated substrate such as a topical swab or pad, that comprises the clay, or a buccal patch or other bioadhesive polymeric compositions comprising the clay. Lozenges are typically discoid-shaped solids containing the clay in a suitably flavored base. The base may be a hard sugar candy, glycerinated gelatin, or the combination of sugar with sufficient gelatin to give it form. Lozenges are placed in the mouth where they slowly dissolve, liberating the clay for direct contact with the affected mucosa. Lozenges can be prepared, for example, by adding water slowly to a mixture of the powdered clay along with excipients such as a sugar and a gum until a pliable mass is formed. The mass is rolled out and the lozenge pieces cut from the flattened mass, or the mass can be rolled into a cylinder and divided. Each cut or divided piece is shaped and allowed to dry, to thus form the lozenge dosage form.

Bioadhesive compositions are particularly suitable for treating proctitis. Patches comprising a polymer and the clay, where the polymer becomes adhesive in the presence of body fluids, are known in the art. Nonlimiting examples of biocompatible polymers that can be used to make a bioadhesive composition include polyethers, such as polyoxyalkylene block copolymers; cellulosic polymers (including hydroxypropylmethyl cellulose, hydroxymethyl cellulose, hydroxypropyl cellulose, methyl cellulose and ethylhydroxyethyl cellulose); gelatin; polyethylene glycol; polyacrylic acid (such as Carbopol™ gel); and glycerol (glycerin). More than one of these exemplary polymers may be included in the composition to provide the desired characteristics and other biocompatible polymers or other additives may also be included in the composition to the extent the inclusion is not inconsistent with performance requirements of the composition.

### Optional Additives

The composition may also include conventional additives such as adhesive agents, antioxidants, crosslinking or curing agents, pH regulators, pigments, dyes, refractive particles, conductive species, antimicrobial agents, active agents and permeation enhancers. In those embodiments wherein adhesion is to be reduced or eliminated, conventional detackifying agents may also be used. These additives, and amounts thereof, are selected in such a way that they do not significantly interfere with the desired chemical and physical properties of the composition.

Non-limiting examples of suitable excipients, diluents, and carriers include: fillers and extenders such as starch, sugars, mannitol, and silicic derivatives; binding agents such as carboxymethyl cellulose and other cellulose derivatives, alginates, gelatin, and polyvinyl pyrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as acetyl alcohol, glycerol monostearate; carriers such as propylene glycol and ethyl alcohol, and lubricants such as talc, calcium and magnesium stearate, and solid polyethyl glycols.

Additives may be present in the compositions, such as flavouring, sweetening or colouring agents, or preservatives. Mint, such as from peppermint or spearmint, cinnamon, eucalyptus, citrus, cassia, anise and menthol are examples of suitable flavouring agents. Flavouring agents are preferably present in the oral compositions in an amount in the range of from 0 to 3%; preferably up to 2%, such as up to 0.5%, preferably around 0.2%, in the case of liquid compositions. Sweeteners include artificial or natural sweetening agents, such as sodium saccharin, sucrose, glucose, saccharin, dextrose, levulose, lactose, mannitol, sorbitol, fructose, maltose, xylitol, thaumatin, aspartame, D-tryptophan, dihydrochalcones, acesulfame, and any combinations thereof, which may be present in an amount in the range of from 0 to 2%, preferably up to 1% w/w, such as 0.05 to 0.3% w/w of the oral composition.

Other optional ingredients of oral aqueous compositions include humectants, surfactants (non-ionic, cationic or amphoteric), thickeners, gums and binding agents. A humectant adds body to the mouthspray formulation and retains moisture in a dentifrice composition. In addition, a humectant helps to prevent microbial deterioration during storage of the formulation. It also assists in maintaining phase stability and provides a way to formulate a transparent or translucent dentifrice. Suitable humectants include glycerine, xylitol, glycerol and glycols such as propylene glycol, which may be present in an amount of up to 50% w/w each, but total humectant is preferably not more than about 60-80% w/w of the composition. For example, liquid compositions may comprise up to about 30% glycerine plus up to about 5%, preferably about 2% w/w xylitol.

When the oral compositions are in the form of a mouthspray, it is preferred to include a film forming agent up to about 3% w/w of the oral composition, such as in the range of from 0 to 0.1%, preferably about 0.001 to 0.01%, such as about 0.005% w/w of the oral composition. Suitable film-formers include (in addition to sodium hyaluronate) those sold under the tradename Gantrez™.

Much of the foregoing description has been primarily directed to the treatment of proctitis. It should be recognized, however, that the same principles discussed above are also generally applicable to treatment of mucosal disorders occurring in other regions of the body, with the product form of the therapeutic composition being modified for administration to the other targeted mucosal site. For example, the therapeutic composition of the present disclosure is applicable for the prevention and/or treatment of mucosal disorders of the vagina, bladder, urethra, and the entire gastrointestinal tract (for example including stomach, small intestine, large intestine and rectum). The mucosal disorders can include but are not limited to inflammatory bowel disease, colitis, cystitis, GERD, proctitis, stomatitis, celiac disease and Crohn's disease. Mucosal disorders at these other locations are mechanistically similar to proctitis, and particularly when the disorder is the result of chemotherapy or radiation therapy. For example, patients undergoing radiation therapy treatment for uterine cancer can develop proctitis as a side effect of treatment. Proctitis in this patient population can impede the progress of cancer treatment. The pharmaceutical substances described above are also applicable for treatment of mucosal disorders in other regions of the body. The method of delivery to the affected region may be by any convenient technique as suitably adapted for the particular region of the body at issue.

The method of delivery to the affected region may be by any convenient technique as suitably adapted for the particular region of the body at issue. Some examples of possible product forms for administration of the therapeutic composition a gel formulated into a suppository would be one preferred product form for administration to treat mucosal surfaces of either the rectum or the vagina. A tablet, patch or film could be formulated to administer the therapeutic composition sublingually. A slurry or oral solution could be used for treatment of mucosal surfaces in the esophagus and/or gastrointestinal tract. A bladder irrigation solution would be administered to the bladder by catheter. A spray would be advantageous in delivering the composition to either the nasal cavity or the lungs, while a droplet formulation would be advantageous for delivery to the eye or inner ear.

### C. Exemplary Dosing Regimens and Patient Populations

In the studies conducted herein, the clay composition was applied topically to the areas of proctitis three times daily. It will be appreciated that this dosing regimen is merely exemplary, and the dosing schedule can be varied according to each individual, to the severity of proctitis, to the area of proctitis, and in accord with other parameters. By way of example, the clay formulation can be applied topically to mucosal surfaces of the oral cavity or gastro-intestinal tract, in some embodiments, one to eight applications per day can begin 24 hours before chemotherapy or radiation, and may continue after the conclusion of cancer treatment or other therapy associated with proctitis. By way of another example, the clay formulation can be administered to the desired local area, one, two, three, four, five or more times per day, and treatment can begin before or concurrent with chemotherapy and/or radiation, and may cease prior to the end of chemotherapy and/or radiation or may continue after chemotherapy and/or radiation has ended.

In one embodiment, the method is for treating or preventing proctitis resulting from radiation or chemotherapy for cancer. The method includes the step of administering to a patient an effective amount of a solution or suspension formed by placing one of the solid dosage forms containing therapeutic agent in an aqueous solution. The solution is administered as, for example, a mouth-rinse. Optionally, additional agents may be present in the solid dosage form.

In another embodiment, the method for treating or preventing proctitis resulting from radiation or chemotherapy for cancer includes the step of administering a solid dosage form described herein to the oral cavity of a patient, for example, sublingually, wherein clay comes into contact with the inflamed tissue.

Treatment according to the disclosed methods can begin prior to cancer treatment or other condition or therapy associated with proctitis (e.g., prophylactically, and/or 1-2 days or up to 1 week prior), at or near the same time as cancer treatment or other therapy associated with proctitis (e.g., simultaneously with, within 1-4 hours of, or on the same day as the treatment), or shortly after the cessation of cancer treatment or other condition or therapy associated with proctitis (e.g., within 1-4 days of cessation, and/or prior to or upon appearance of symptoms). Treatment can then be maintained, for example, until any symptoms of proctitis have substantially cleared or the risk of developing such symptoms has passed. Thus, treatment started before or at or near the same time as cancer treatment or other condition or therapy associated with proctitis can be maintained, e.g., for 1-3, e.g., 1-2 days. In other examples, treatment is maintained for 1-4 or 2-3 weeks following the cessation of cancer treatment or other therapy associated with proctitis, as determined to be appropriate by one of skill in the art. In specific examples, the treatment according to the present disclosure is carried out only prior to cancer treatment or other therapy associated with proctitis (such as treatment of HIV/AIDS with chemotherapeutic agents or with antiretroviral agents with or without chemotherapeutic agents); prior to and concurrently with cancer treatment or other therapy associated with proctitis; prior to, concurrently with, and after cessation of cancer treatment or other therapy associated with proctitis; concurrently with cancer treatment or other therapy associated with proctitis only; concurrently with and after cessation of cancer treatment or other therapy associated with proctitis only; after cessation of cancer treatment or other therapy associated with proctitis only; or prior to and after cessation of cancer treatment or other therapy associated with proctitis only. Further, treatment according to the methods of the present disclosure can be altered, stopped, or re-initiated in a patient, depending on the status of any symptoms of proctitis. Treatment can be carried out at intervals determined to be appropriate by those of skill in the art. For example, the administration can be carried out 1, 2, 3, 4 or more times/day. It will be appreciated that the patients to be treated with the methods described herein are not limited to cancer patients, but include any patient that is at risk of or has developed proctitis, including HIV/AIDS patients being treated chemotherapeutic agents with or without antiretroviral agents.

Chemotherapeutic agents likely to cause proctitis include but are not limited to anthracyclines (such as daunorubicin, doxorubicin, pirubicin, idarubicin and mitoxantrone), methotrexate, dactinomycin, bleomycin, vinblastine, cytarabin, fluorouracil, mitramycine, etoposide, floxuridine, 5-fluorouracil, hydroxyurea, methotrexate, mitomycin, vincristine, vinorelbine, taxanes (such as docetaxel and paclitaxel), ifosfamide/eoposide, irinotecan, platinum, as well as combinations including one or more of these drugs. The risk of developing proctitis is markedly exacerbated when chemotherapeutic agents that typically produce mucosal toxicity are given in high doses, in frequent repetitive schedules, or in combination with ionizing irradiation (e.g., conditioning regimens prior to bone marrow transplant). The lesions induced by chemotherapeutic agents are clinically significant by about a week after treatment and the severity progresses to about day ten through twelve and begins to subside by day fourteen. Accordingly, in some embodiments, the patient to be treated is one undergoing or scheduled to undergo treatment with one or more of these chemotherapeutic agents.

Non-therapeutic radiation and/or chemical exposure, as may happen from accidents, acts of war, acts of civilian terrorism, space flights, or rescue and clean-up operations can also result in proctitis. In these scenarios the effects of radiation in the hematopoietic system and the gastrointestinal tract are critical. Furthermore, inflammation can be caused by medications, or conditions such as dietary protein deficiency, anal sex, or by conditions that affect the entire body, such as infections, allergic reactions, toxic plants or radiation therapy.

### Combination Regimens and Combination Compositions

The methods presently disclosed can be used alone or in conjunction with other approaches to reducing the severity of proctitis. For example, the disclosed methods can be carried out in combination with antimicrobial or antifungal therapies, e.g., therapies involving administration of antibiotics such as nystatin, amphotericin, acyclovir, valacyclovir, clotimazole, and fluconazole. As a specific example of such treatment, patients with pelvic cancer receiving radiotherapy may have colonization of the vaginal region with certain bacteria. Selective decontamination of the vaginal cavity with anti-microbial agents has the benefit of reducing proctitis associated with radiation therapy, but there may be limitations to the beneficial effects of such treatment. Anti-microbial therapy can kill bacteria, but cannot reduce endotoxin, and indeed may actually increase endotoxin. As endotoxin is a potent mediator of inflammation, it may contribute to the aggravation of proctitis and, thus, co-treatment with an antiendotoxin compound (e.g., a Lipid A analog, such as eritoran) and antibiotics can be used as a more effective approach to treating proctitis in such patients, according to the present disclosure.

The methods presently disclosed can also be used in conjunction with palliative therapies including the use of topical rinses, gels, or ointments that include lidocaine, articaine, and/or morphine, as well as other analgesic or anti-inflammatory agents. Specific examples of other agents and approaches that can be used in combination with TLR4 antagonists, according to the methods presently disclosed, include the following: palifermin (recombinant keratinocyte growth factor; rHuKGF; Kepivance™; Amgen) and AES-14 (uptake-enhanced L-glutamine suspension) (Peterson, J. Support Oncol. 4(2 Suppl. 1)9-13, 2006); oral cryotherapy, low-level laser therapy, chiorhexidine, amifostine, hematologic growth factors, pentoxifylline, and glutamine (Saadeh, Pharmacotherapy 25(4):540-554, 2005); amifostine, antibiotic paste or pastille, hydrolytic enzymes, ice chips, benzydamine, calcium phosphate, honey, oral care protocols, povidone, and zinc sulphate (Worthington et al., Cochrane Database Syst. Rev. 2:CD000978, 2006); flurbiprofen (e.g., administered as a tooth patch; Stokman et al., Support Care Cancer 13(1):42-48, 2005); diphenhydramine, magnesium hydroxide/aluminum hydroxide, nystatin, and corticosteroids (Chan et al., J. Oncol. Pharm. Pract. 11(4):139-143, 2005); oral transmucosal fentanyl citrate (e.g., administered in the form of a lozenge; Shaiova et al., Support Care Cancer 12(4):268-273, 2004); clonazepam (e.g., in the form of a tablet; Gremeau-Richard et al., Pain 108(102):51-57, 2004); capsaicin (e.g., in the form of a lozenge; Okuno et al., J. Cancer lntegr. Med. 2(3):179-183, 2004); ketamine (e.g., in the form of an oral rinse; Slatkin et al., Pain Med. 4(3):298-303, 2003); and granulocyte-macrophage colony-stimulating factor (GM-CSF)/granulocyte colony-stimulating factor (G-CSF), laser light therapy, and glutamine supplements (Duncan etal., Aliment. Pharmacol. Ther. 18(9):853-874, 2003).

An exemplary assay for the treatment of proctitis may be performed as described in the phase 3 clinical trial of Kepivance™ (palifermin) (see, Spielberger, N. Engl. J. Med., 351(25):2590-2598 (2004)), or as described in phase II clinical trials of GM-CSF (molgramostin) (see McFleese et al., Br. J. Radiol. 79(943):608-13 (2006)).

In some embodiments, the patient is not undergoing, or has not undergone treatment with vitamin D.

In some embodiments of the present disclosure, the clay may be combined with a second therapeutic agent. In one embodiment, the second therapeutic agent is a pain reliever or anaesthetic, such as an anaesthetic found in an enema or suppository (e.g., phenol, benzocaine, phenazone, antipyrine, analgesine, dyclonine hydrochloride salt). In some embodiments, the pain reliever acts as a bactericidal and fungicidal in addition to acting as a local anaesthetic. In some embodiments, the pain reliever is an anaesthetic selected from the group consisting of fentanyl, hexylresorinol, dyclonine hydrochloride, asbenzocaine and phenol. For example, fentanyl has been approved for topical administration (transdermal patch), and is often used in cancer for pain control. In some embodiments, the clay is combined with fentanyl.

The Toll-like receptor (TLR) family plays a fundamental role in pathogen recognition and activation of innate immunity. Toll-like receptor 4 is a protein that in humans is encoded by the TLR4 gene. The TLR4 receptor detects lipopolysaccharide (LPS) from Gram-negative bacteria and is thus important in the activation of the innate immune system. TLR4 has also been designated as CD284 (cluster of differentiation 284). Examples of TLR4 agonists are: morphine, morphine-3-glucuronide, oxycodone, levorphanol, pethidine, fentanyl, methadone and buprenorphine.

According to the methods disclosed herein, a TLR4 antagonist can be administered to a patient before, during, and/or after treatment with a therapy that causes proctitis or puts the patient at risk of developing such proctitis. As is noted above, such treatments include radiation and chemotherapy, which act by blocking the growth of rapidly dividing cells, such as cancer cells and epithelial cells that line the surfaces of the gastrointestinal, respiratory, and genitourinary tracts. Specific examples of treatments that can lead to proctitis include radiation treatment (e.g., pelvic, whole body, targeted, and/or hyperfractionated radiation), as well as chemotherapeutic regimens used in the treatment of, or as adjuvant treatments for, conditions such as breast cancer, colon cancer, gastric cancer, genitourinary (e.g., bladder, prostate, or testicular) cancer, gynecologic (e.g., cervical, endometrial, ovarian, or uterine) cancer, head and neck/esophageal cancer, leukemia, lung (small cell or non small-cell) cancer, lymphoma (Hodgkin's or non-Hodgkin's), melanoma, multiple myeloma, pancreatic cancer, and sarcoma.

### III. EXAMPLES

The following examples are given to illustrate the present disclosure. It should be understood that the claims are not to be limited to the specific conditions or details described in the examples.

### Example 1

### Acute Radiation Model of Proctitis

An acute radiation model in rats has proven to be an accurate, efficient and cost-effective technique to provide a preliminary evaluation of anti-proctitis compounds (Murphy, C.K. et al., (2008) Clin Cancer Res. 1(14):4292-7). The course of proctitis in this model is well-defined and scores approximately 3 days following radiation. The acute model has little systemic toxicity, resulting in few animal deaths, thus permitting the use of smaller groups for initial efficacy studies. It has also been used to study specific mechanistic elements in the pathogenesis of proctitis. Molecules that show efficacy in the acute radiation model may be further evaluated in the more complex models of fractionated radiation, chemotherapy, or concomitant therapy.

### Animals

Male Sprague-Dawley rats (Harlan Laboratories), aged 5 to 6 weeks, with average body weight of 217 g at study commencement, were used. Animals were individually numbered using an ear punch and housed in small groups of approximately 5 animals per cage. Animals were acclimatized prior to study commencement. During this period of 3 days, the animals were observed daily in order to reject animals that presented in poor condition.

### Housing and Diet

The study was performed in animal rooms provided with filtered air at a temperature of 70° F.+/−5° F. and 50%+/−20% relative humidity. Animal rooms were set to maintain a minimum of 12 to 15 air changes per hour. The room was on an automatic timer for a light/dark cycle of 12 hours on and 12 hours off with no twilight. The lights turned on at 6 am and off at 6 pm. Bed-O-Cobs® bedding was used. Bedding was changed a minimum of once per week. Cages, tops, bottles, etc. were washed with a commercial detergent and allowed to air dry. A commercial disinfectant was used to disinfect surfaces and materials introduced into the hood. Floors were swept daily and mopped a minimum of twice weekly with a commercial detergent. Walls and cage racks were sponged a minimum of once per month with a dilute bleach solution. A cage card or label with the appropriate information necessary to identify the study, dose, animal number and treatment group marked all cages. The temperature and relative humidity was recorded during the study, and the records retained.

Animals were fed with a Purina Labdiet® 5053 rodent diet, and water was provided ad libitum.

### Animal Randomization and Allocations.

Animals were randomly and prospectively divided into three (3) treatment groups prior to irradiation. Each animal was identified by an ear punch corresponding to an individual number. For more consistent identification, ear punch numbering was used rather than tagging, since tags may become dislodged during the course of the study. A cage card was used to identify each cage or label marked with the study number, treatment group number and animal numbers.

### Weights and Survival

All animals were weighed daily and their survival recorded, in order to assess possible differences in animal weight among treatment groups as an indication for proctitis severity and/or possible toxicity resulting from the treatments.

### Clay Composition

A dioctahedral smectite, calcium aluminosilicate clay with an average particle size of less than about 80 microns was mixed with deionized water to form a suspension of 10% w/v clay in water.

### Proctitis Induction

Twenty-five (25) male Sprague-Dawley rats were prospectively randomized into two treatment groups of ten (10) animals each and a control group of five (5) animals. On study day 0, twenty rats were anesthetized with ketamine (100 mg/kg) and xylazine (5 mg/kg) and placed beneath 4 mm lead shields with their lower abdomens exposed. Proctitis was induced on Day 0 by a single acute dose of radiation of 20 Gy directed to the lower abdomen. A group of 5 animals served as un-irradiated controls. All animals were assessed visually daily for the presence of diarrhea and/or bloody stool and body weights were measured once daily from Day −3 to Day 10. On days 3, 7, and 10 proctitis severity was assessed in all animals using video endoscopy, and images were taken and proctitis severity scored by a blinded observer.

Radiation was generated with a Kimtron 160 kilovolt potential (15-ma) source at a focal distance of 30 cm, hardened with a 0.35 mm Cu filtration system. Irradiation targeted the rectal mucosa with a single exposure of the lower pelvic region at a rate of 1 Gy/minute with the rest of the body being shielded. Prior to irradiation, animals were anesthetized with an intraperitoneal injection of ketamine (100 mg/kg) and xylazine (5 mg/kg). Animals were inclined slightly in a downward position, to allow gravity to move some of the other abdominal organs out of the way of the radiation path.

TABLE 4
Study Design

| | | | | | Video | |
|---|---|---|---|---|---|---|
| | Number of | | | Dosing | Endoscopy | |
| Group | Animals | Radiation | Treatment | Schedule | Schedule | Volume |
| 1 | 5 | NO | Untreated | — | Days | — |
| | | | | | 3, 7, 10 | |
| 2 | 10 | 20 Gy | Vehicle-WFI | Days 0 | Days | 0.5 mL/Dose |
| | | Day 0 | (intrarectal) | to 10* | 3, 7, 10 | |
| | | | | (BID) | | |
| 3 | 10 | 20 Gy | 15% clay | Days 0 | Days | 0.5 mL/Dose |
| | | Day 0 | (intrarectal) | to 10* | 3, 7, 10 | |
| | | | | (BID) | | |

*The first dose of clay on Day 0 was administered 1-2 hours following radiation.

### Exampel 2

### Proctitis Evaluation—Video Endoscopy

All animals underwent video endoscopy on days 3, 7, and 10 to assess the severity of proctitis in each treatment group. The mean proctitis scores for all treatment groups on the individual days are shown in FIGS. 3-5. Proctitis scores were significantly elevated in the radiation group treated with only vehicle compared to the untreated control group on all days of endoscopy. Treatment with 15% clay substantially reduced the extent of proctitis, especially apparent on Day 10, however this improvement just missed reaching statistical significance (p=0.059).

Specifically, on day 3, the untreated control group had an average proctitis score of 0.0, while the radiation-treated control group had an average score of 1.4. The group treated with 15% clay had an average score of 1.1. On day 7, the untreated control group had an average proctitis score of 0.0, while the radiation-treated control group had an average score of 2.0 and the group treated with 15% clay had an average score of 1.7. On day 10, the untreated control group maintained an average proctitis score of 0.0, while the radiation-treated control group had an average score of 2.2 and the group treated with 15% clay had an average score of 1.6. Significant improvements in the treatment groups were assessed through t-test comparison to the radiation treated vehicle control group. FIG. 6 simply shows all endoscopy scores from the three treatment groups on each day of the study.

### Example 3

### Evaluation of Proctitis in Animals Treated with Clay Composition

A composition comprising clay was given intrarectally (i.r.) twice daily (b.i.d.) from day 0 to day 10 while another group was administered water for injection (WFI) for the same schedule, as indicated in Table 4. As a control, animals in Group I received a saline vehicle twice daily at the same dose volume as the clay compositions. The clay selected for use in the study was a calcium-based aluminosilicate clay, which is essentially non-swelling in aqueous fluids. Tissue was taken for possible histological evaluation on Day 10. The weight and general health of the animals were evaluated daily.

To evaluate proctitis severity, animals were anesthetized with an inhalation anesthetic, and underwent video endoscopy of the lower colon on days 3, 7, and 10. On Day 10, all animals were euthanized by CO₂ asphyxiation, death was confirmed by monitoring heartbeat in accordance with USDA guidelines, and the colon and rectal tissues were dissected and fixed in neutral buffered formalin. Proctitis was scored visually by comparison to a validated photographic scale, ranging from 0 for normal, to 4 for severe ulceration (clinical scoring). In descriptive terms, this proctitis scoring scale is defined in Table 5, below.

TABLE 5
Endoscopy Colitis Scoring Scale

| Score: | Description: |
|---|---|
| 0 | Normal |
| 1 | Loss of vascularity |
| 2 | Loss of vascularity and friability |
| 3 | Friability and erosions |
| 4 | Ulcerations and bleeding |

### Weight Change (FIGS. 1 and 2)

The mean daily percent weight gains for all treatment groups are shown in FIG. 1. The unirradiated control group gained an average of 23.3% of their starting weight during the study. The vehicle control group (Treatment Group 2) gained only an average of 4.0% of their starting weight during the study, while the group treated with clay had gained 3.5% of their starting weights by the conclusion of the study Day 10.

To evaluate the significance of these differences, the mean area under the curve (AUC) was calculated for each animal from the percent weight gain data, and the means and standard errors were plotted (FIG. 2). The weight loss from each treatment group was compared to the radiated vehicle-treated group using Student's t-tests. As expected, there was a statistically significant increase in weight change between the un-irradiated controls and the vehicle-treated radiation control group (p<0.001). There was no statistically significant difference between the group treated with clay and the vehicle control group (p=0.599).

### Example 4

Video EndoscopyThe treatment responses of the colon/rectum to the radiation and clay treatment is readily apparent in the images presented in FIG. 7. The corresponding endoscopy scores for the images presented in FIG. 7 are as follows: untreated control—score of 0; radiation vehicle control—score of 2, inflammation, altered vascularity and friability; clay—score of 1, some altered vascularity.

### Diarrhea (FIG. 8).

The percentage of animals exhibiting diarrhea on each study day for all treatment groups are shown in FIG. 8. The vehicle control group exhibited peak levels of diarrhea on Day 7, with 40% of the animals exhibiting diarrhea. Levels of diarrhea were notably lower in the clay treatment group, as diarrhea was only exhibited by 20% of the animals in this group from Days 5 to 7.

### Overall:

No adverse reactions to treatment with clay, given twice daily, were observed over the duration of the study.

As anticipated, the radiation had a significant negative impact on the weight gain of all groups receiving radiation. There was a statistically significant increase in weight change between the non-irradiated controls and the vehicle-treated radiation control group. There was no statistically significant difference in weight change in the group that received 15% clay as compared to the radiation control group.

Radiation induced significant levels of proctitis in the vehicle control group when compared to the untreated control group on all days of endoscopy. Treatment with 15% clay via intrarectal administration resulted in near significant improvements in proctitis severity.

The vehicle control group exhibited peak levels of diarrhea on Days 7 and 8 (40 and 30% of animals, respectively). Levels of diarrhea were notably lower in the clay treatment group, as peak levels reached only 20% of the animals in this group from Days 5 to 7.

### Example 5

### Binding of the Pro-Inflammatory Cytokine with a Clay Composition

A dioctahedral smectite, calcium aluminosilicate clay with an average particle size of less than about 80 microns was suspended in phosphate buffered saline at six different concentrations: 0.5 mg/mL, 1 mg/mL, 5 mg/mL, 10 mg/mL, 50 mg/mL, and 100 mg/mL.

Recombinant TNFa (50 mg/ml stock in 100% ddH₂O) was added to each of the clay samples to a final concentration of 1000 pg/mL TNFa. One sample of 1000 pg/mL TNFα in 100% PBS with no clay was prepared as a control. The samples were vortexed for 30 seconds and allowed to incubate at room temperature for 30 minutes. During this incubation the samples were vortexed again every 10 minutes for 5 seconds. After incubation, the samples were centrifuged at 10,000 rpm for 5 minutes and the supernatant was isolated.

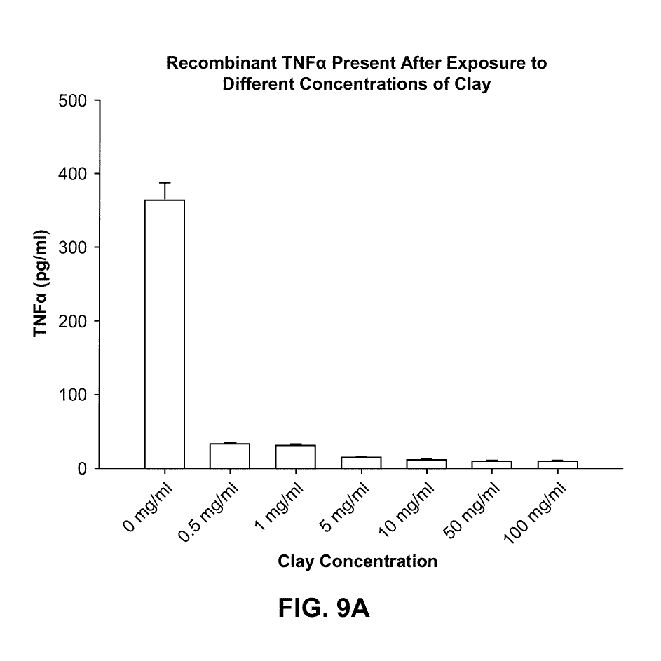

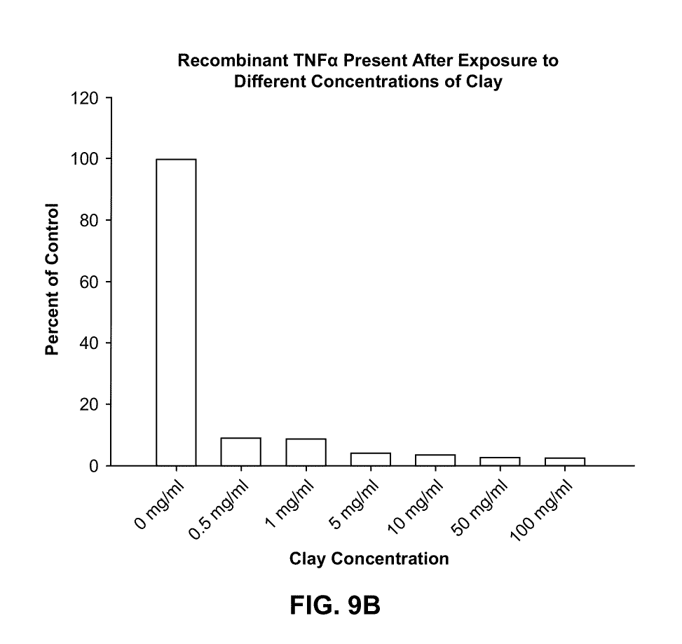

Follow the protocol on a TNFa ELISA kit (R&D Systems Inc.), the amount of TNF-alpha in each supernatant was examined. The results are shown in FIGS. 9A-9B.

While a number of exemplary aspects and embodiments have been discussed and illustrated, those of skill in the art will recognize certain modifications, permutations, additions and sub-combinations thereof which do not depart from the scope of the present disclosure. It is to be understood that the disclosure is not to be limited to the specific embodiments disclosed herein, as such are presented by way of example. It is therefore intended that the following appended claims and claims hereafter introduced are interpreted to include all such modifications, permutations, additions and sub-combinations as are within their true spirit and scope.

All literature and similar materials cited in this application, including, but not limited to, patents, patent applications, articles, books, treatises, internet web pages and other publications cited in the present disclosure, regardless of the format of such literature and similar materials, are expressly incorporated by reference in their entirety for any purpose to the same extent as if each were individually indicated to be incorporated by reference. In the event that one or more of the incorporated literature and similar materials differs from or contradicts the present disclosure, including, but not limited to defined terms, term usage, described techniques, or the like, the present disclosure controls.

It is claimed:

1. A method for treating, preventing, reducing severity of, or reducing or delaying the onset of proctitis, comprising:
administering to a subject a therapeutically effective amount of a composition comprising a clay.

2. The method of claim 1, wherein the clay is a low-swelling or a non-swelling, calcium clay species.

3. The method of claim 1, wherein the clay is a smectite clay.

4. The method of claim 1, wherein the administering comprises administering a composition comprising calcium montmorillonite clay.

5. The method of claim 4, wherein the montmorillonite clay comprises a majority of particles with a dry state fractionation size of between about 50-200 μm.

6. The method of claim 4, wherein the montmorillonite clay comprises a majority of particles with a wet state fractionation size of less than 2 μm.

7. The method of claim 1, wherein the clay has a selected average particle size that is achieved by sieving or by air classification of the clay.

8. The method of claim 4, wherein the montmorillonite clay is non-swelling and a calcium species as evidenced by a shrink/swell potential (SV) of less than 1.5 COLE index value.

9. The method of claim 4, wherein the montmorillonite clay exhibits an extractable bases value for calcium of greater than 90 mEq/100 g clay, when extracted using ammonium acetate, or a value for calcium of between 6-12 mEq/100 g clay/L when extracted with deionized water.

10. The method of claim 1, wherein the administering comprises administering to a subject with cancer, lower-gastrointestinal mucositis, radiation-induced proctitis, chemotherapy-induced proctitis, or at risk of developing proctitis.

11. The method of claim 1, wherein the administering comprises administering to a subject undergoing or planning to undergo chemotherapy.

12. The method of claim 10, wherein the administering is prior to or concurrent with initiation of radiation therapy in the cancer subject.

13. The method of claim 1, wherein the administering is after radiation therapy.

14. The method of claim 1, wherein the administering continues for the duration of radiation therapy.

15. The method of claim 1, wherein the administering comprises administering at least once daily.

16. The method of claim 1, wherein the administering comprises administering rectally a fluid comprising the clay.

17. The method of claim 16, wherein the fluid is a solution, a suspension, a paste, or a gel.

18. The method of claim 16, wherein administering comprises administering the fluid wherein the fluid is held in the rectum for a recommended period of time.

19. The method of claim 1, wherein the administering comprises administering a composition comprising a polymer.

20. The method of claim 1, wherein the administering comprises administering a solid dosage form that disintegrates in an aqueous medium.

21. The method of claim 1, wherein the subject is concurrently treated with at least one therapeutic agent.

22. The method of claim 21, wherein the therapeutic agent is a pain reliever, a chemotherapeutic, an anti-inflammatory or antibiotic.

23. The method of claim 1, further comprising, prior to said administering, contacting a clay with a fluid to form a composition suitable for rectal or oral administration.
